(19) United States
(12) Patent Application Publication
(10) Pub. No.: US 2011/0263984 A1
(43) Pub. Date: Oct. 27, 2011

(54) ADAPTIVE CLUTTER FILTERING IN AN ULTRASOUND SYSTEM

(75) Inventors: Moo Ho BAE, Seoul (KR); Young Seok LEE, Chuncheon-si (KR); Sung Bae PARK, Chuncheon-si (KR); Kyoung Bo LEE, Hanam-si (KR)

(73) Assignee: SAMSUNG MEDISON CO., LTD.

(21) Appl. No.: 13/094,452

(22) Filed: Apr. 26, 2011

(30) Foreign Application Priority Data
Apr. 26, 2010 (KR) 10-2010-0038422
Mar. 3, 2011 (KR) 10-2011-0018707

(51) Int. Cl. A61B 8/00; G06F 15/00

(52) U.S. Cl. 600/453; 702/191

(57) ABSTRACT

Embodiments for performing clutter filtering in an ultrasound system are disclosed. In one embodiment, an ultrasound data acquisition unit acquires ultrasound data from a target object for color Doppler imaging and a processing unit extracts a plurality of frequency components of the ultrasound data using an autoregressive model and computes a mean frequency component of the plurality of frequency components. The processing unit detects frequency components corresponding to a clutter signal based on the plurality of frequency components and the mean frequency component and performs clutter filtering upon the ultrasound data by using the frequency components corresponding to the clutter signal.

### CROSS-REFERENCE TO RELATED APPLICATIONS

The present application claims priority from Korean Patent Application Nos. 10-2010-0038422 and 10-2011-0018707 filed on Apr. 26, 2010 and Mar. 3, 2011, the entire subject matter of which is incorporated herein by reference.

### TECHNICAL FIELD

The present disclosure generally relates to strain imaging, and more particularly to adaptive clutter filtering in an ultrasound system.

### BACKGROUND

An ultrasound system has become an important and popular diagnostic tool since it has a wide range of applications. Specifically, due to its non-invasive and non-destructive nature, the ultrasound system has been extensively used in the medical profession. Modern high-performance ultrasound systems and techniques are commonly used to produce two or three-dimensional images of internal features of an object (e.g., human organs).

Generally, the ultrasound system may operate in a Brightness-mode (B-mode) visualizing a reflectivity of an ultrasound signal reflected from a target object as a 2-dimensional image, a Doppler mode visualizing a velocity of a moving object as spectral Doppler by using the Doppler effect, a color Doppler mode visualizing velocity and direction of a moving object with colors by using the Doppler effect and an elasticity mode visualizing mechanical characteristics of tissues such as the elasticity of the same.

In the color Doppler mode, the ultrasound system may transmit an ultrasound signal to the target object and receive the ultrasound echoes to thereby form a Doppler signal. The ultrasound system may form a color Doppler image based on the Doppler signal. The Doppler signal may include a low frequency signal (the so-called clutter signal) due to the motion of a cardiac wall or valve of a heart and a noise in addition to a signal caused by a blood flow (referred to as “blood flow signal”). The clutter signal may have amplitude, which is over 100 times than that of the blood flow signal. The clutter signal may be an obstacle to accurately detect a velocity of the blood flow. Thus, it is required to remove the clutter signal from the Doppler signal to accurately detect the velocity of the blood flow.

A frequency down mixing method has been used to remove the clutter signal. According to the frequency down mixing method, frequency components corresponding to the clutter signal are estimated and then down mixing, i.e., frequency shifting, is performed upon the Doppler signal such that a center frequency of the clutter signal becomes zero. Thereafter, the clutter filtering is performed to remove the clutter signal.

Generally, the ultrasound system may acquire an amount of the ultrasound data by the ensemble number and estimate the frequency components corresponding to the clutter signal by using the acquired ultrasound data. However, it may be difficult to accurately estimate the frequency components corresponding to the clutter signal and frequency components corresponding to the blood flow signal by using the ultrasound data corresponding to the limited ensemble number.

To cope with the above problem, the conventional clutter filtering has been performed by setting a high cutoff frequency of a high pass filter. When the cutoff frequency is set high, a Doppler signal (i.e., blood flow signal) corresponding to a blood flow of a relatively low speed may be removed and a clutter signal of a relatively high frequency may not be removed. Thus, the motion of the blood flow may not be accurately indicated on a color Doppler image.

### SUMMARY

Embodiments for adaptively performing clutter filtering in an ultrasound system are disclosed herein. In one embodiment, by way of non-limiting example, an ultrasound system comprises: an ultrasound data acquisition unit configured to perform a transmit/receive operation including transmitting ultrasound signals to a target object and receiving ultrasound echoes reflected from the target object to thereby acquire ultrasound data for color Doppler imaging; and a processing unit configured to extract a plurality of frequency components of the ultrasound data using an autoregressive model and compute a mean frequency component of the plurality of frequency components, the processing unit being further configured to detect frequency components corresponding to a clutter signal based on the plurality of frequency components and the mean frequency component and perform clutter filtering upon the ultrasound data by using the frequency components corresponding to the clutter signal.

In another embodiment, a method of performing cluttering filtering in an ultrasound system, comprises: a) performing a transmit/receive operation including transmitting ultrasound signals to a target object and receiving ultrasound echoes reflected from the target object to thereby acquire ultrasound data for color Doppler imaging; b) extracting a plurality of frequency components of the ultrasound data using an autoregressive model; c) detecting a mean frequency component of the plurality of frequency components; d) detecting frequency components corresponding to a clutter signal based on the plurality of frequency components and the mean frequency component; and e) performing clutter filtering upon the ultrasound data by using the frequency components corresponding to the clutter signal.

The Summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 is a block diagram showing an illustrative embodiment of an ultrasound system.

FIG. 2 is a schematic diagram showing an example of displaying a brightness mode image and a region of interest.

FIG. 3 is a block diagram showing an illustrative embodiment of an ultrasound data acquisition unit.

FIG. 4 is a flowchart showing an illustrative embodiment of forming a color Doppler image.

FIG. 5 is a flowchart showing an illustrative embodiment of detecting the frequency components corresponding to the clutter signal.

### DETAILED DESCRIPTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

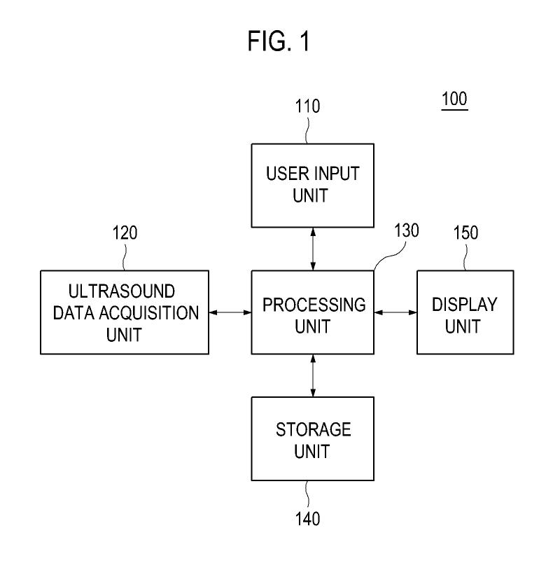

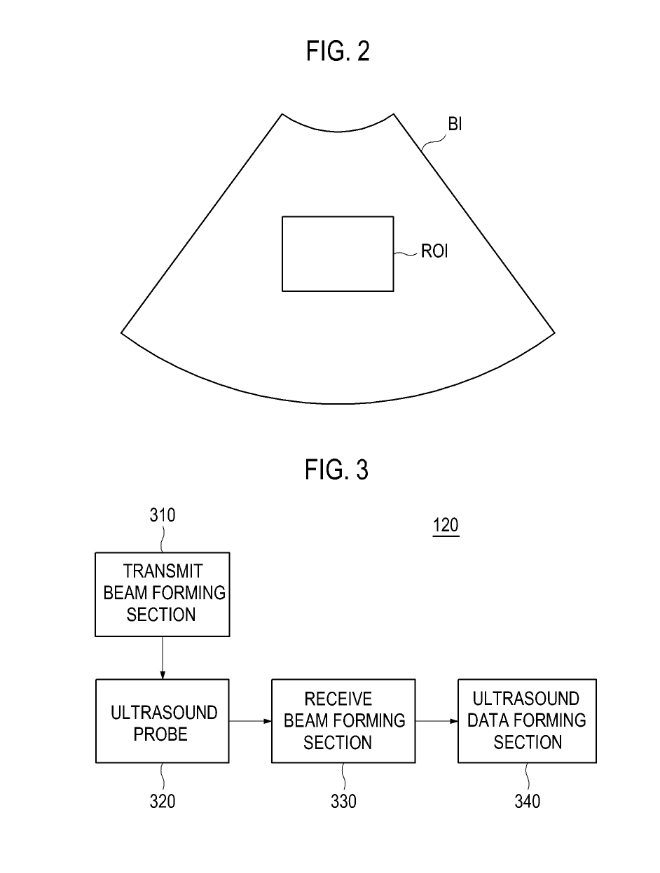

Referring to FIG. 1, an ultrasound system constructed in accordance with one embodiment is shown. The ultrasound system 100 may include a user input unit 110, an ultrasound data acquisition unit 120, a processing unit 130, a storage unit 140 and a display unit 150. The user input unit 110 may receive input information of a user. The input information may include input information for setting a region of interest (ROI) on a brightness (B)-mode image BI, as illustrated in FIG. 2. The ROI may include a color box for acquiring ultrasound data for color Doppler imaging. However, the ROI may not be limited thereto. The user input unit 100 may be a control panel, a mouse, a keyboard, a track ball and the like.

The ultrasound data acquisition unit 120 may be configured to transmit ultrasound beams to a target object and receive ultrasound echoes reflected from the target object to thereby form ultrasound data representative of the target object. An operation of the ultrasound acquisition unit will be described in detail by referring to FIG. 3.

FIG. 3 is a block diagram showing an illustrative embodiment of the ultrasound data acquisition unit 120. Referring to FIG. 3, the ultrasound data acquisition unit 120 may include a transmit (Tx) signal forming section 310. The Tx signal forming section 310 may generate a plurality of Tx signals. The Tx signal forming section 310 may form a Tx pattern of the Tx signals according to image modes such as a brightness mode, a Doppler mode, a color Doppler mode and the like. In one embodiment, the Tx pattern of the Tx signals may include a first Tx pattern for the brightness mode and a second Tx pattern based on an ensemble number for the color Doppler mode. The ensemble number represents the number for transmission/reception of ultrasound signals along one single scan line to acquire Doppler signals.

The ultrasound data acquisition unit 120 may further include an ultrasound probe 320, which is coupled to the Tx signal forming section 210. The ultrasound probe 320 may include an array transducer containing a plurality of transducer elements for reciprocal conversion between electric signals and ultrasound signals. The ultrasound probe 320 may be configured to transmit ultrasound signals in response to the Tx signals. In one embodiment, the transmitted ultrasound signals may include first ultrasound signals based on the first Tx pattern of the Tx signals and second ultrasound signals based on the second Tx pattern of the Tx signals. The ultrasound probe 320 may be further configured to receive ultrasound echoes reflected from the target object to thereby output receive signals. In one embodiment, the receive signals may include first receive signals associated with the first ultrasound signals and second receive signals associated with the second ultrasound signals. The ultrasound probe 320 may include a convex probe, a linear probe and the like.

The ultrasound data acquisition unit 120 may further include a beam forming section 330, which is coupled to the ultrasound probe 320. The beam forming section 330 may be configured to digitize the receive signals into digital signals. The beam forming section 330 may be configured to apply delays to the digital signals in consideration of distances between the elements of the ultrasound probe 320 and focal points. The beam forming section 330 may further sum the delayed digital signals to form receive-focused signals. In one embodiment, the beam forming section 330 may form first receive-focused signals based on the first receive signals and second receive-focused signals based on the second receive signals.

The ultrasound data acquisition unit 120 may further include an ultrasound data forming section 340, which is coupled to the beam forming section 330. The ultrasound data forming section 340 may be configured to form ultrasound data based on the receive-focused signals. In one embedment, the ultrasound data forming section 340 may form first ultrasound data for a B-mode image BI. The first ultrasound data may be radio frequency data, although it is not limited thereto. The ultrasound data forming section 340 may form second ultrasound data corresponding to the ROI for a Color Doppler image (i.e., ensemble data). The second ultrasound data may include in-phase/quadrature data, although the second ultrasound data may not be limited thereto.

Referring to FIG. 1, the processing unit 130, which is coupled to the ultrasound data acquisition unit 120, may be embodied with at least one of a central processing unit, a microprocessor, a graphic processing unit and the like. However, the processing unit 130 may not be limited thereto.

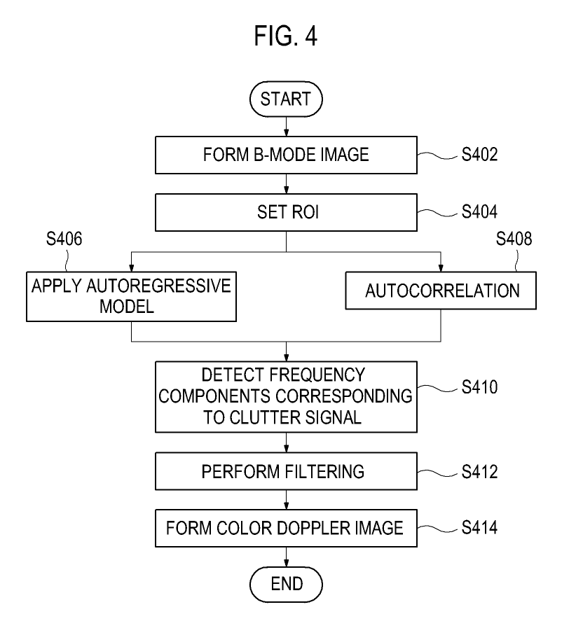

FIG. 4 is a flowchart showing an illustrative embodiment of forming a color Doppler image. Referring to FIG. 4, the processing unit 130 may form a B-mode image BI based on the first ultrasound data, which are provided from the ultrasound data acquisition unit 120 at S402. The B-mode image BI may be displayed on the display unit 150 for allowing a user to set the ROI thereon by using the user input unit 110.

If the input information is provided through the user input unit 110, then the processing unit 130 may set the ROI on the B-mode image based on the input information at S404. In response to setting the ROI, the processing unit 130 may control the ultrasound data acquisition unit 120 for operation in the color Doppler mode to thereby obtain the second ultrasound data from the ROI (i.e., ensemble data).

The processing unit 130 may be configured to estimate a plurality of frequency components and first strengths (or powers) of the second ultrasound data by using the autoregressive (AR) model at S406. Generally, a function H(z) of an m^{th} order AR model may be expressed as the following equation.

$$H ( z ) = \frac{\sqrt{e}}{1 + {a}_{2} {z}^{- 1} + … + {a}_{( p + 1 )} {z}^{- m}} \qquad ( 1 )$$

wherein a numerator may be represented by a minimized dispersion e and poles, which are roots of a denominator, may be represented by a polynomial of linear prediction coefficients aₖ and z.

Linear prediction may be adopted to estimate the linear prediction coefficient aₖ of equation (1). The linear prediction is a technique that estimates a current value from linear sum of previous values of a given signal. Assuming that N discrete signals (xₙ)_{nε[0,N]} are provided, forward linear prediction yₙ and backward linear prediction zₙ may be indicated as linear prediction coefficients (aₙ)_{nε[1,k]} of k coefficients.

$${y}_{n} = - {\sum }_{i = 1}^{k} {a}_{i} {x}_{n - i} \\ {z}_{n} = - {\sum }_{i = 1}^{k} {a}_{i} {x}_{n + i} \qquad ( 2 )$$

The forward linear prediction yₙ may be represented by the minimized sum Fₖ of squared errors, as follows.

$${F}_{k} = {\sum }_{n = k}^{N} {( {x}_{n} - {y}_{n} )}^{2} = {\sum }_{n = k}^{N} {( {x}_{n} - ( - {\sum }_{i = 1}^{k} {a}_{i} {x}_{n - i} ) )}^{2} \qquad ( 3 )$$

Typically, the linear prediction coefficients (aₙ)_{nε[1,N]} may be selected through minimization of the sum of squared errors. The backward linear prediction zₙ may be represented by the minimized sum Bₖ of squared errors, as follows.

$${B}_{k} = {\sum }_{n = k}^{N} {( {x}_{n} - {z}_{n} )}^{2} = {\sum }_{n = k}^{N} {( {x}_{n} - ( - {\sum }_{i = 1}^{k} {a}_{i} {x}_{n - i} ) )}^{2} \qquad ( 4 )$$

To estimate the linear prediction coefficients (aₙ)_{nε[1,N]} for minimizing the error of the forward linear prediction or the backward linear prediction, initial state parameters may be stabilized by the Burg's recursion based on the Levinson-Durbin recursion.

If the linear prediction coefficients of the AR model, in which the initial state parameters may be stabilized by the Burg's recursion based on the Levinson-Durbin recursion, are estimated, then all poles of the denominator in equation (1) may be estimated.

In one embodiment, the processing unit 130 may be configured to detect two poles for the second ultrasound data by using the second order AR model and detect frequency components and strengths corresponding to the respective two poles. In one embodiment, the AR model may not be limited to the second order AR model. The second order AR model may be defined as follows.

$$H ( z ) = \frac{{z}^{2} \sqrt{e}}{{z}^{2} + {a}_{1} z + {a}_{2}} = \frac{{z}^{2} \sqrt{e}}{( z - {p}_{1} ) ( z - {p}_{2} )} \qquad ( 5 )$$

wherein p₁ represents a first pole of the second order AR model function H(z) and p₂ represents a second pole of the second order AR model function H(z).

The processing unit 130 may be further configured to detect frequency components ω₁ and ω₂ corresponding to the respective two poles of the second ultrasound data as the following equation.

$${ω}_{1} = {tan}^{- 1} ( \frac{Im ( {p}_{1} )}{Re ( {p}_{1} )} )$$

$${ω}_{2} = {tan}^{- 1} ( \frac{Im ( {p}_{2} )}{Re ( {p}_{2} )} )$$

Further, the processing unit 130 may be further configured to compute a mean frequency component ω₃ and a second strength (or power) corresponding to the second ultrasound data by using auto-correlation at S408. In another embodiment, the processing unit 130 may be configured to compute a mean frequency component ω₃ and a second strength (or power) corresponding to the second ultrasound data by using the fast Fourier transform.

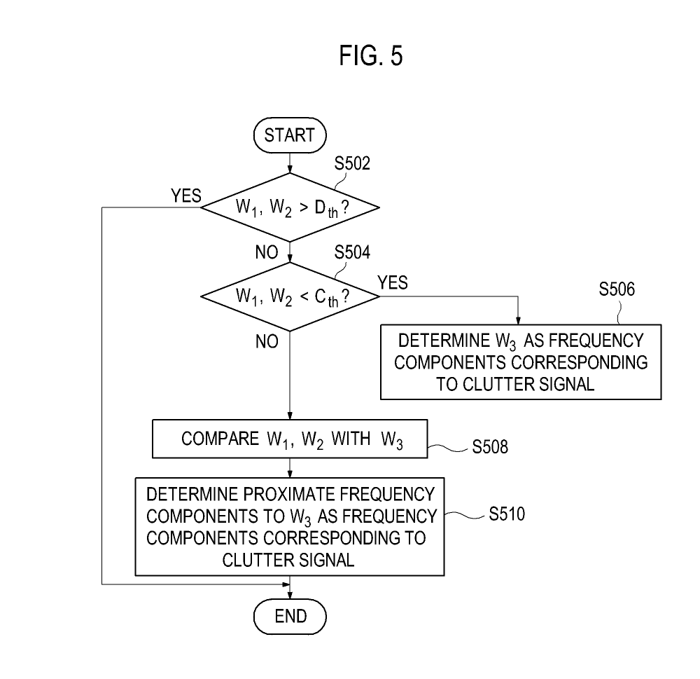

The processing unit 130 may be configured to detect frequency components corresponding to the clutter signal by using the frequency components ω₁ and ω₂ and the mean frequency component ω₃ at S410. The step of S410 will be described in detail by referring to FIG. 5.

FIG. 5 is a flowchart showing an illustrative embodiment of detecting the frequency components corresponding to the clutter signal. Referring to FIG. 5, the processing unit 130 may be configured to compare the frequency components ω₁ and ω₂, which have been detected by using the second order AR model, with a predetermined Doppler threshold Dₜₕ at S502. In such a case, the Doppler threshold Dₜₕ may be an arbitrary value estimated by using a plurality of clinical data and set to different values according to parts of organs in the target object.

If it is determined that the frequency components ω₁ and ω₂ are greater than the Doppler threshold Dₜₕ at S502, then the processing unit 130 may be configured to determine that the clutter signal does not exist in the Doppler signal. This is so that the detection of the frequency components corresponding to the clutter signal may not be carried out.

On the other hand, if it is determined that at least one of the frequency components ω₁ and ω₂ is less than the Doppler threshold Dₜₕ at S502, then the processing unit 130 may be configured to compare the frequency components ω₁ and ω₂ with a predetermined clutter threshold Cₜₕ at S504. If it is determined that the frequency components ω₁ and ω₂ are less than the clutter threshold Cₜₕ at S502, then the frequency components ω₁ and ω₂ may be considered as a noise and a clutter signal. This is so that the processing unit 130 may be configured to detect the mean frequency component ω₃ as the frequency component corresponding to the clutter signal at S506.

However, if it is determined that at least one of the frequency components ω₁ and ω₂ is greater than the Clutter threshold Cₜₕ at S504, then the processing unit 130 may be configured to compare the frequency components ω₁ and ω₂ with the mean frequency component ω₃ at S508. The processing unit 130 is further configured to detect proximate frequency components from the mean frequency component ω₃ as the frequency components corresponding to the clutter signal at S510.

Referring back to FIG. 4, the processing unit 130 is configured to perform filtering upon the second ultrasound data by using the frequency components corresponding to the clutter signal at S412. In one embodiment, the processing unit 130 may be configured to set the frequency components corresponding to the clutter signal as the frequency down mixing frequencies. The processing unit 130 may be further configured to perform the frequency down mixing upon the second ultrasound data based on the down mixing frequencies. The processing unit 130 may be configured to restore the clutter-filtered second ultrasound data to the original frequencies.

Further, if the frequency components corresponding to the clutter signal are not detected, then the processing unit 130 may be configured to compare the first strengths and the second strengths for the second ultrasound data to perform noise removal upon the second ultrasound data. The noise removal may be performed by using a well-known method so that the detailed description thereof will be omitted herein. The processing unit 130 may be configured to form a color Doppler image by using the second ultrasound data with the clutter signal filtered at S414.

Referring to FIG. 1, the storage unit 140, which is coupled to the ultrasound data acquisition unit 120 via the processing unit 130, is configured to store the ultrasound data (first ultrasound data and second ultrasound data) acquired in the ultrasound data acquisition unit 120. Also, the storage unit 140 may be configured to store the Doppler threshold Dₜₕ and the clutter threshold Cₜₕ. Further, the storage unit 140 may further include the second ultrasound data with the clutter signal filtered.

The display unit 150 may display the B-mod image and the color Doppler image, which have been formed in the processing unit 130. The display unit 150 may include at least one of a cathode ray tube (CRT) display, a liquid crystal display (LCD), an organic light emitting diode (OLED) display and the like.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound system, comprising:
an ultrasound data acquisition unit configured to perform a transmit/receive operation including transmitting ultrasound signals to a target object and receiving ultrasound echoes reflected from the target object to thereby acquire ultrasound data for color Doppler imaging; and
a processing unit configured to extract a plurality of frequency components of the ultrasound data using an autoregressive model and compute a mean frequency component of the plurality of frequency components, the processing unit being further configured to detect frequency components corresponding to a clutter signal based on the plurality of frequency components and the mean frequency component and perform clutter filtering upon the ultrasound data by using the frequency components corresponding to the clutter signal.

2. The ultrasound system of claim 1, wherein the processing unit is configured to extract the plurality of frequency components corresponding to a plurality of poles from the ultrasound data by using the autoregressive model

3. The ultrasound system of claim 2, wherein the processing unit is configured to:
compare each of the plurality of frequency components with a predetermined Doppler threshold,
compare, when at least one of the frequency components is less than the predetermined Doppler threshold, each of the plurality of frequency components with a predetermined clutter threshold, and
compare, when at least one of the frequency components is greater than the predetermined clutter threshold, each of the plurality of frequency components with the mean frequency component to detect frequency components proximate to the mean frequency component as the frequency components corresponding to the clutter signal.

4. The ultrasound system of claim 3, wherein the processing unit is configured to set, when the frequency components are greater than the predetermined clutter threshold, the mean frequency component as the frequency component corresponding to the clutter signal.

5. The ultrasound system of claim 1, wherein the processing unit is configured to:
set the frequency components corresponding to the clutter signal as frequency down mixing frequencies,
perform frequency down mixing upon the ultrasound data based on the frequency down mixing frequencies, and
perform the clutter filtering upon the frequency down mixed ultrasound data.

6. A method of performing cluttering filtering in an ultrasound system, comprising:
a) performing a transmit/receive operation including transmitting ultrasound signals to a target object and receiving ultrasound echoes reflected from the target object to thereby acquire ultrasound data for color Doppler imaging;
b) extracting a plurality of frequency components of the ultrasound data using an autoregressive model;
c) detecting a mean frequency component of the plurality of frequency components;
d) detecting frequency components corresponding to a clutter signal based on the plurality of frequency components and the mean frequency component; and
e) performing clutter filtering upon the ultrasound data by using the frequency components corresponding to the clutter signal.

7. The method of claim 6, wherein the step b) includes extracting the plurality of frequency components corresponding to a plurality of poles from the ultrasound data by using the autoregressive model.

8. The method of claim 7, wherein the step d) includes:
d1) comparing each of the plurality of frequency components with a predetermined Doppler threshold;
d2) comparing, when at least one of the frequency components is less than the predetermined Doppler threshold, each of the plurality of frequency components with a predetermined clutter threshold; and
d3) comparing, when at least one of the frequency components is greater than the predetermined clutter threshold, each of the plurality of frequency components with the mean frequency component to detect frequency components proximate to the mean frequency component as the frequency components corresponding to the clutter signal.

9. The method of claim 8, wherein the step d) comprises setting, when the frequency components are greater than the predetermined clutter threshold, the mean frequency component as the frequency component corresponding to the clutter signal.

10. The method of claim 6, wherein the step e) includes:
setting the frequency components corresponding to the clutter signal as frequency down mixing frequencies,
performing frequency down mixing upon the ultrasound data based on the frequency down mixing frequencies, and
performing the clutter filtering upon the frequency down mixed ultrasound data.
